(19) United States
(12) Patent Application Publication
(10) Pub. No.: US 2020/0029546 A1
(43) Pub. Date: Jan. 30, 2020

(54) BUG TERMINATOR SYSTEMS

(71) Applicant: Frank Aylward, Toronto (CA)

(72) Inventor: Frank Aylward, Toronto (CA)

(21) Appl. No.: 16/100,746

(22) Filed: Aug. 10, 2018

(30) Foreign Application Priority Data
Jul. 26, 2018 (CA) 3012500

(51) Int. Cl. A01M 1/20; B05B 12/12; B05B 12/02; B65D 83/26; B05B 11/00; A61L 9/14

(52) U.S. Cl. CPC A01M 1/2038; B05B 12/122; B05B 12/02; A61L 2209/134; B05B 11/3053; A61L 9/14; A61L 2209/111; B65D 83/262

(57) ABSTRACT

The bug terminator system includes a bug terminator assembly including a docking station having a housing with at least one container-receiver, a logic circuit, at least one electromechanical valve, necessary electrical circuitry, a powerer, a motor, an actuator, at least one inputting means, and at least one pressurized-container for removably containing a dispensable liquid to control pests and alternately to dispense room fresheners or other liquids in an atomized form.

### INDEX TO RELATED APPLICATIONS

This Application claims benefit under 35 USC 119 of Canada Patent Application 3,012,500 filed Jul. 26, 2018 the disclosure of which is incorporated herein be reference in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### TECHNICAL FIELD

The present invention relates generally to the field of fluid sprinkling, spraying and diffusing of existing art and more specifically relates to dispensing via a bug terminator system.

### RELATED ART

Insects and bugs play a role within our natural environments. Many times these insects and bugs can become pests and spread diseases when they interact with humans and animals. The insects and bugs may need to be controlled via pesticides, especially when their populations increase. Use of pesticides can be expensive and if not properly dispensed may harm unintended organisms. Bugs may be especially annoying and cause damage if left unchecked within interior spaces. A suitable solution is desired.

U.S. Pat. No. 8,091,734 to Paul E Furner et al., relates to a compact spray device. The described compact spray device includes a power source which is provided to a dispensing unit that includes a housing having an aerosol container disposed therein. A sleep period interval between spray operations is selected and a motion sensor is activated to detect motion within a sensory path of the sensor after completion of the sleep period interval. If no motion is detected by the motion sensor a fluid is automatically discharged from the aerosol container and the sleep interval is reset and if motion is detected by the motion sensor a delay time interval is initiated and fluid is not discharged from the aerosol container. The dispensing unit alternates between activating the sensor to detect motion and resetting the delay time interval until no motion is detected by the motion sensor at the expiration of the delay time interval, which results in the automatic discharge of fluid and a resetting of the sleep period interval.

### SUMMARY OF THE INVENTION

In view of the foregoing disadvantages inherent in the known fluid sprinkling, spraying and diffusing means art, the present disclosure provides a novel bug terminator system. The general purpose of the present disclosure, which will be described subsequently in greater detail, is to provide an automatic pesticide, insecticide, repellant, poison, air freshener, disinfectant or other spray-type delivery system for automating the delivery of these solutions to desired areas.

A bug terminator system is disclosed herein. The bug terminator system includes a bug terminator assembly including a docking station having a housing with at least one container-receiver, a logic circuit, at least one electromechanical valve, necessary electrical circuitry, a powerer, a motor, an actuator, at least one inputting means, and at least one pressurized-container for removably containing a dispensable liquid. Solar panels may be in the top of the device for charging purpose. A hook may be mounted on the top of the unit.

The bug terminator assembly comprises in functional combination the docking station and the at least one pressurized-container; the docking station able to control timed and volumetric dispensing of the dispensable liquid via inputted commands according to user-determined programming inputted via the at least one inputting means. The housing of the docking station houses the at least one container-receiver, the logic circuit, the at least one electromechanical valve, the electrical circuitry, the motor, the actuator, and the at least one inputting means. Each at least one container-receiver is configured to removably receive each respective at least one pressurized-container. The logic circuit, the at least one electromechanical valve, the motor, and the actuator are provided power via the powerer via the electrical circuitry. The motor is able to actuate the actuator and the at least one electromechanical valve to allow the dispensing of the dispensable liquid for the at least one pressurized-container through the at least one container-receiver and into a surrounding ambient environment for control of bugs and pests. A second canister may dispense an insect attractant. In operation, in a cycle, the attractant would be dispensed and then shortly after, the pesticide or the like is dispensed. This may be repeated every 10-20 minutes or whatever duration is best-suited. This is in an effort to reduce the area covered by the insecticide for health reasons, and to reduce the amount required, thus reducing the canister size and/or extending the refill time.

According to another embodiment, a method of use for the bug terminator system is also disclosed herein. The method includes providing a bug terminator system, programming the bug terminator system, inserting at least one pressurized-container into a corresponding at least one container-receiver, and powering the bug terminator system for use. The method may further comprise the steps of: setting a timer to a desired spray-timing, and enabling use of a motion sensor for sensing unauthorized entry into a specified area.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a bug terminator system, constructed and operative according to the teachings of the present disclosure.

FIG. 1 is a perspective view of the bug terminator system during an ‘in-use’ condition, according to an embodiment of the disclosure.

FIG. 2 is a perspective view of the bug terminator system of FIG. 1, according to an embodiment of the present disclosure.

FIG. 3 is a perspective view of the bug terminator system of FIG. 1, according to an embodiment of the present disclosure.

FIG. 4 is a perspective view of the bug terminator system of FIG. 1, according to an embodiment of the present disclosure.

FIG. 5 is a flow diagram illustrating a method of use for the bug terminator system, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to fluid sprinkling, spraying and diffusing and more particularly to a bug terminator system as used to improve the efficient and effective control of pests.

Generally, the present invention comprises an automatic pesticide, insecticide, repellant, poison, air freshener, disinfectant or other spray-type delivery system for automating the delivery of these dispensable liquid solutions to areas. The device preferably includes a logic circuit (or other suitably equivalent controlling means) and mechanisms containing a circuit that will enable a user to program the device to discharge a spray of poison, disinfecting or air freshening spray(s) at a pre-selected time interval or day/time interval chosen in the unit's memory. An electromechanical valve opens to let contents escape when the spraying mechanism of the system is activated. The valve connects to a pressurized spray cylinder and it releases a controlled atomized burst of insecticide, pesticide, repellant, disinfectant, and or air freshener as programmed. Control circuits allow the user to set when the system goes ‘off’. The system also includes a motion sensor that can detect entry to the space by people or insects and then automatically release the contents. The device can be plugged in to a standard electrical outlet or be battery operated. The present invention allows for use when occupants are asleep or not at home. It automatically controls the amount of spray so to conserve. Further, it can be timed for nighttime use or activated by motion sensor and is refillable. When used it reduces bugs and/or odor in all spaces by using different chemicals depending on what is active in the environment.

The following is a list of components: 1) on/off switch; 2) power supply; 3) positive lead to switch; 4) unit ground; 5) pump unit; 6) repellant canister(s); 7) Carbon dioxide cannister(s); 8) charger contact and harness; 9) A/C cord or USB; 10) A/B outlets; 11) base/charger; 12) logic module and harness with timer; 13) hinges A/B to access to refill product; and housing cover release.

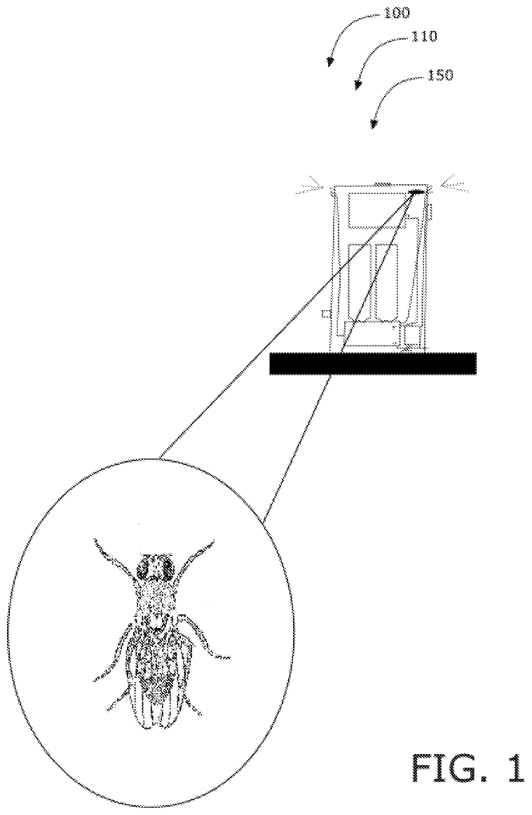

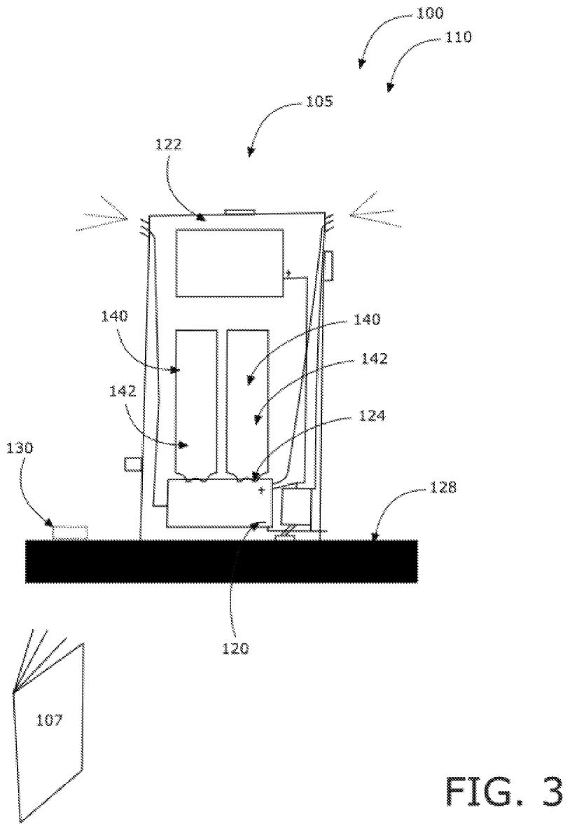

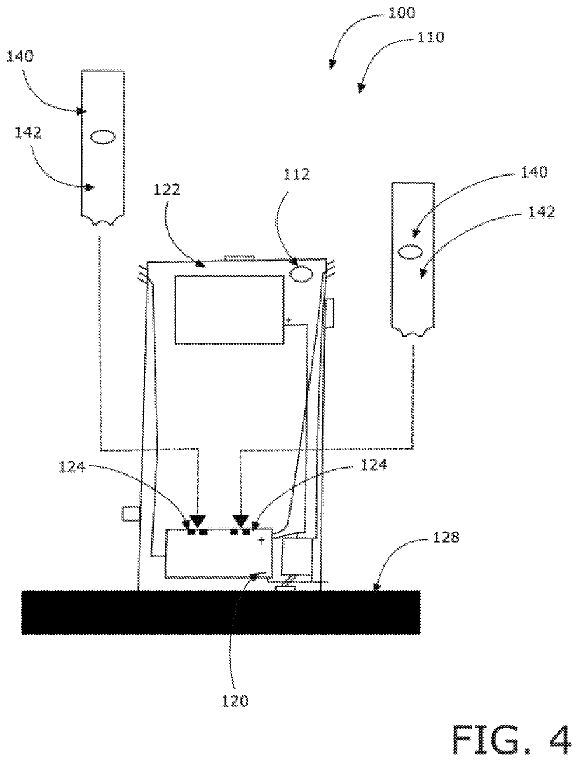

Referring now more specifically to the drawings by numerals of reference, there is shown in FIGS. 1-4, various views of a bug terminator system 100.

FIG. 1 shows a bug terminator system 100 during an ‘in-use’ condition 150, according to an embodiment of the present disclosure. Here, the bug terminator system 100 may be beneficial for use by a user to control pests and/or odors present in an inner room. As illustrated, the bug terminator system 100 may include a bug terminator assembly 110 including a docking station 120 having a housing 122 with at least one container-receiver 124, a logic circuit, at least one electromechanical valve, necessary electrical circuitry, a powerer, a motor 128, a pump; an actuator, at least one inputting means 130, and at least one pressurized-container 140 for removably containing a dispensable liquid 142. Those with ordinary skill in the art will now appreciate that upon reading this specification and by their understanding the art of powering, communicating signals, use of logic controllers, microchips, pumping, valving, etc as described herein, methods of dispensing via controlled means will be understood by those knowledgeable in such art. In embodiments with two of the pressurized-containers 140 one may serve to spray attractant and the other after a timed interval spray the pesticide to efficiently attract and kill pests.

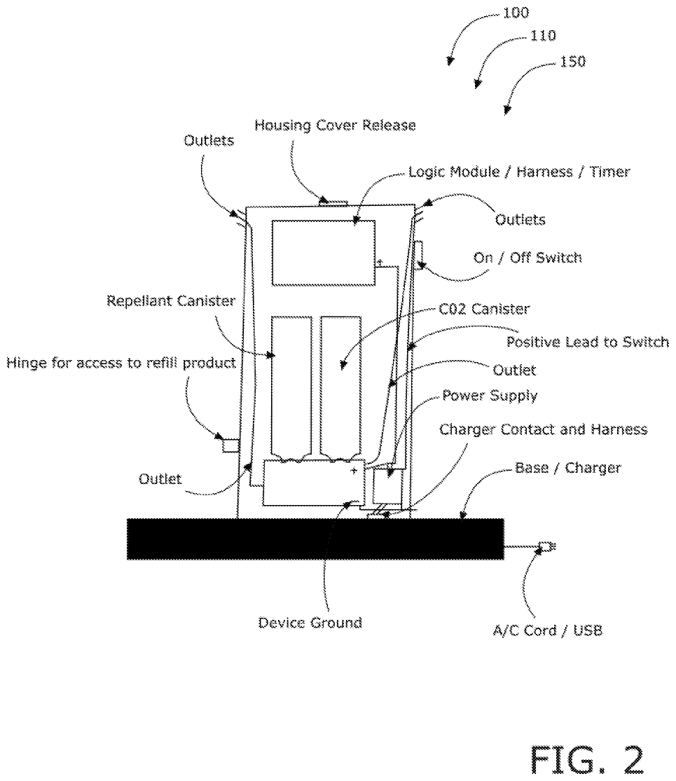

FIGS. 2-4 show various views of the bug terminator system 100 of FIG. 1, according to an embodiment of the present disclosure. As above, the bug terminator system 100 may include the bug terminator assembly 110 comprising in functional combination the docking station 120 and the at least one pressurized-container 140; the docking station 120 able to control timed and volumetric dispensing of the dispensable liquid 142 via inputted commands according to user-determined programming inputted via the at least one inputting means 130 (buttons, switches, wireless control and communication, rheostats, knobs and the like). The housing 122 of the docking station 120 houses the at least one container-receiver 124, the logic circuit, the at least one electromechanical valve, the electrical circuitry, the motor 128, the actuator, and the at least one inputting means 130. Each at least one container-receiver 124 (two shown) is configured to removably receive each respective at least one pressurized-container 140 (two shown). The logic circuit, the at least one electromechanical valve, the motor 128, and the actuator are provided power via the powerer via the electrical circuitry such that the device can be operated and when shut down, not operated. The motor 128 is able to actuate the actuator and the at least one electromechanical valve to allow the dispensing of the dispensable liquid 142 for the at least one pressurized-container 140 through the at least one container-receiver 124 and out into a surrounding ambient environment for control of bugs and pests (odor and the like). The at least one pressurized-container 140 (shown engaged vertically) is sealingly received by the at least one container-receiver 124 such that bug terminator system 100 does not leak liquid/fluid (dispensable fluid 142) during non-use and that the dispensable fluid 142 is properly directed. Sealing means may comprise o-ring seals, gaskets or other suitably equivalent sealing means. The bug terminator assembly 110 may further comprise a motion sensor 112 as a means for detecting entry into a specific area (such as bugs flying or crawling).

The at least one pressurized-container 140 is able to be (manually) refilled with the dispensable liquid 142 when the dispensable liquid 142 is depleted. The at least one pressurized-container 140 (two shown) each preferably comprise a siteglass such that a volume of the dispensable liquid 142 can be visually determined and refilled as needed. The dispensable liquid 142 may be propelled via a propellant; the dispensable liquid 142 able to be atomized for introduction into the surrounding air. The propellant may or may not comprise aerosol. Compressed air such as CO₂ or the like may be used.

The dispensable liquid 142 may comprise insecticide; repellent; disinfectant; pesticide; poison; air-freshener or the like. Upon reading this specification, it should be appreciated that, under appropriate circumstances, considering such issues as user preferences, design preference, structural requirements, marketing preferences, cost, available materials, technological advances, etc., other fluid solutions such as, for example, other fluids, solutions, non-fluids, air, etc., may be sufficient. The bug terminator system 100 may further comprise timing means such as a timer. The powerer may comprise batteries or connection means to an ac electrical source that runs power through a cord.

According to one embodiment, the bug terminator system 100 may be arranged as a kit 105. In particular, the bug terminator system 100 may further include a set of instructions 107. The instructions 107 may detail functional relationships in relation to the structure of the bug terminator system 100 such that the bug terminator system 100 can be used, maintained, or the like, in a preferred manner.

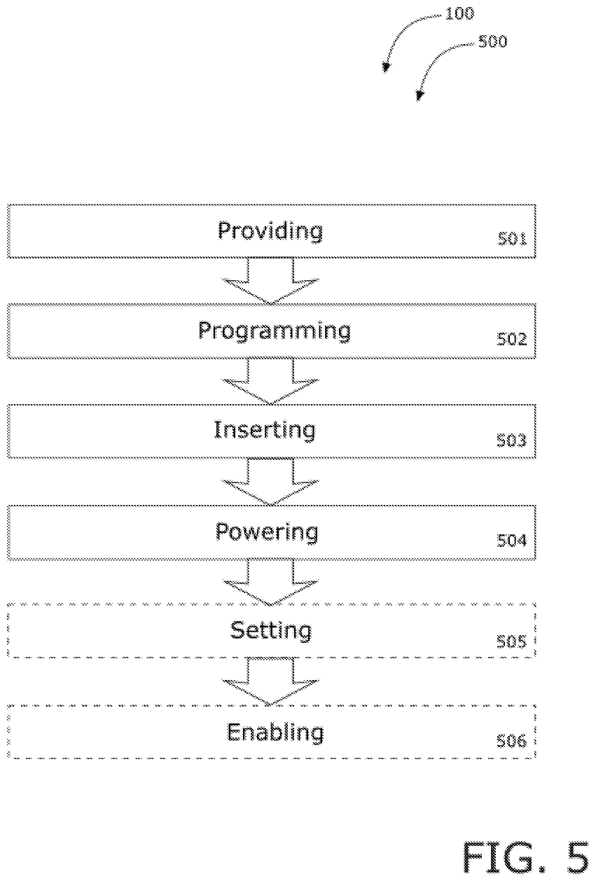

FIG. 5 is a flow diagram illustrating a method of use 500, according to an embodiment of the present disclosure. In particular, the method of use 500 for the bug terminator system 100 may include one or more components or features of the bug terminator system 100 as described above. As illustrated, the method of use 500 may include the steps of: step one 501, providing a bug terminator system; step two 502, programming the bug terminator system; step three 503, inserting at least one pressurized-container into a corresponding at least one container-receiver, and step four 504 powering the bug terminator system for use. The method may further comprise the steps of: 505 setting a timer to a desired spray-timing, and step six 506 enabling use of a motion sensor for sensing unauthorized entry into a specified area.

It should be noted that steps 505 and 506 are optional steps and may not be implemented in all cases. Optional steps of method of use 500 are illustrated using dotted lines in FIG. 5 so as to distinguish them from the other steps of method of use 500. It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for bug and pest control and room freshening are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A bug terminator system comprising:
a bug terminator assembly including
a docking station having
a housing with
at least one container-receiver;
a logic circuit;
at least one electromechanical valve;
electrical circuitry;
a powerer;
a motor;
a pump;
an actuator;
at least one inputting means; and
at least one pressurized-container for removably containing a dispensable liquid;
wherein said bug terminator assembly comprises in functional combination said docking station and said at least one pressurized-container, said docking station able to control timed and volumetric dispensing of said dispensable liquid via inputted commands according to user-determined programming inputted via said at least one inputting means;
wherein said housing of said docking station houses said at least one container-receiver, said logic circuit, said at least one electromechanical valve, said electrical circuitry, said motor, said actuator, and said at least one inputting means;
wherein each said at least one container-receiver is configured to removably receive each said at least one pressurized-container;
wherein said logic circuit, said at least one electromechanical valve, said motor, and said actuator are provided power via said powerer via said electrical circuitry; and
wherein said motor is able to actuate said actuator and said at least one electromechanical valve to allow said dispensing of said dispensable liquid to be pumped via said pump for said at least one pressurized-container through said at least one container-receiver and into a surrounding ambient environment for control of bugs and pests.

2. The bug terminator system of claim 1, further comprising a motion sensor.

3. The bug terminator system of claim 2, wherein said at least one pressurized-container is able to be refilled with said dispensable liquid when said dispensable liquid is depleted.

4. The bug terminator system of claim 3, wherein the dispensable liquid is propelled via a propellant.

5. The bug terminator system of claim 3, wherein the dispensable liquid comprises insecticide.

6. The bug terminator system of claim 3, wherein the dispensable liquid comprises repellent.

7. The bug terminator system of claim 3, wherein the dispensable liquid comprises disinfectant.

8. The bug terminator system of claim 3, wherein said dispensable liquid comprises pesticide.

9. The bug terminator system of claim 3, wherein said dispensable liquid comprises poison.

10. The bug terminator system of claim 3, wherein said dispensable liquid comprises air-freshener.

11. The bug terminator system of claim 3, wherein the propellant comprises aerosol.

12. The bug terminator system of claim 1, further comprising a timer.

13. The bug terminator system of claim 1, wherein the powerer comprises batteries.

14. The bug terminator system of claim 1, wherein the powerer comprises ac electrical source.

15. The bug terminator system of claim 1, wherein the at least one pressurized-container comprises a siteglass such that a volume of said dispensable liquid can be visually determined.

16. The bug terminator system of claim 1, wherein the at least one pressurized-container is sealingly received by said at least one container-receiver.

17. A bug terminator system comprising:
a bug terminator assembly including
a docking station having
a housing with
at least one container-receiver;
a logic circuit;
at least one electromechanical valve;
electrical circuitry;
a powerer;
a motor;
a pump;
an actuator;
at least one inputting means;
a timer; and
a motion sensor for sensing unauthorized entry into a specified area;
at least one pressurized-container for removably containing a dispensable liquid;
wherein said bug terminator assembly comprises in functional combination said docking station and said at least one pressurized-container, said docking station able to control timed and volumetric dispensing of said dispensable liquid via inputted commands according to user-determined programming inputted via said at least one inputting means;
wherein said housing of said docking station houses said at least one container-receiver, said logic circuit, said at least one electromechanical valve, said electrical circuitry, said motor, said actuator, and said at least one inputting means;
wherein each said at least one container-receiver is configured to removably receive each said at least one pressurized-container;
wherein said at least one pressurized-container is able to be refilled with said dispensable liquid when said dispensable liquid is depleted;
wherein a first said at least one pressurized-container is able to dispense attractant and a second said at least one pressurized-container is able to dispense a pesticide via said pump powered by said motor at a delayed timed interval;
wherein the at least one pressurized-container comprises a siteglass such that a volume of said dispensable liquid can be visually determined;
wherein said logic circuit, said at least one electromechanical valve, said motor, and said actuator are provided power via said powerer via said electrical circuitry;
wherein the powerer comprises ac electrical source; and
wherein said motor is able to actuate said actuator and said at least one electromechanical valve to allow said dispensing of said dispensable liquid for said at least one pressurized-container through said at least one container-receiver and into a surrounding ambient environment for control of bugs and pests.

18. The bug terminator system of claim 17, further comprising set of instructions; and
wherein the bug terminator system is arranged as a kit.

19. A method of use for the bug terminator system, the method comprising the steps of:
providing a bug terminator system;
programming the bug terminator system;
inserting at least one pressurized-container into a corresponding at least one container-receiver; and
powering said bug terminator system for use.

20. The method of claim 19, further comprising the steps of:
setting a timer to a desired spray-timing; and
enabling use of a motion sensor for sensing unauthorized entry into a specified area.
